(19) United States
(12) Patent Application Publication
(10) Pub. No.: US 2025/0325500 A1
(43) Pub. Date: Oct. 23, 2025

(54) USE OF 5-METHOXY-2-AMINOINDAN ("MEAI") IN METHODS FOR TREATING COCAINE ADDICTION

(71) Applicants: Clearmind Medicine Inc., Tel Aviv (IL); BAR-ILAN UNIVERSITY, Ramat Gan (IL)

(72) Inventors: Adi ZULOFF-SHANI, Tel Aviv (IL); Gal Moshe YADID, Shoham (IL)

(21) Appl. No.: 18/869,965

(22) PCT Filed: May 31, 2023
(86) PCT No.: PCT/IB2023/055591

§ 371 Date: Nov. 27, 2024

Related U.S. Application Data
(60) Provisional application No. 63/365,627, filed on Jun. 1, 2022.

(51) Int. Cl. A61K 31/135; A61P 25/36

(52) U.S. Cl. CPC A61K 31/135; A61P 25/36

(57) ABSTRACT

The present disclosure relates to methods of treating cocaine addiction by administrating 5-methoxy-2-aminoindan, or a salt thereof, to a subject in need thereof in a therapeutically effective amount. The disclosure also relates to methods for treating cocaine addiction by administering 5-methoxy-2-aminoindan or a pharmaceutically acceptable salt thereof, and an N-acylethanolamine or a pharmaceutically acceptable salt thereof, to a subject in need thereof in a therapeutically effective amount.

### CROSS-REFERENCE TO RELATED APPLICATION

This application claims priority to U.S. Provisional Application No. 63/365,627, filed on Jun. 1, 2022, the contents of which are hereby incorporated by reference.

### FIELD OF THE DISCLOSURE

In some embodiments, the present invention relates to methods for treating cocaine addiction by administering a therapeutically effective amount of 5-methoxy-2-aminoindan (“MEAI”). In certain other embodiments, the methods of treatment comprise administering MEAI in combination with N-acylethanolamines, for example, palmitoylethanolamide (“PEA”).

### BACKGROUND OF THE DISCLOSURE

Cocaine enhances monoamine neurotransmitter (dopamine, norepinephrine, and serotonin) activity in the central and peripheral nervous systems by blocking the presynaptic reuptake pumps (transporters) for these neurotransmitters. The major synaptic effect of cocaine is the release of dopamine from the synaptic vesicles and the blocking of dopamine reuptake resulting in an enhanced dopaminergic neurotransmission. Thus, cocaine addiction has been termed a disease of the brain's dopamine reward system. Cocaine also has a second action of blocking voltage-gated membrane sodium ion channels. This action accounts for its local anesthetic effect and may contribute to cardiac arrhythmias. https://www.sciencedirect.com/topics/agricultural-and-biological-sciences/cocaine#:˜:text=The%20major%20synaptic%20effect%20of,the%20brain's%20dopamine%20reward%20system.

It's estimated that globally around 0.9% of the population had a drug use (excluding alcohol) disorder in 2017. The trends in prevalence across the world are shown in the chart. At the country-level, this prevalence ranged from 0.4 to 3.5 percent. The highest prevalence was in the United States where around 1-in-30 had a drug use addiction in 2017. When these trends are broken down by age, we see that globally, adults in their twenties are most likely to have a drug use disorder; more than 2 percent (1-in-50) people aged 20-29 do. In the United States, 8-9 percent of adults in their early twenties had a drug use disorder in 2017; this is around 1-in-11 or 1-in-12. It's estimated that globally around 71 million people had a drug use disorder in 2017. The main groups of illicit drugs used in international statistics are opioids, cocaine, amphetamines and cannabis. https://ourworldindata.org/illicit-drug-use

Compounds derived from 2-aminoindan have been shown to selectively bind to the dopamine D3 receptor. U.S. Pat. No. 5,708,018 discloses some 2-aminoindan derivatives and hypothesizes that these 2-aminoindan derivatives may be useful in treating CNS disorders associated with dopamine D3 receptor. One such compound is 5-methoxy-2-aminoindan (“MEAI”).

N-acylethanolamines (NAEs) are lipid-derived signaling molecules. They are formed when one of several types of acyl groups is linked to the nitrogen atom of ethanolamine. Examples of N-acylethanolamines include anandamide (the amide of arachidonic acid (20:4 omega-6) and ethanolamine), N-Palmitoylethanolamine (the amide of palmitic acid (16:0) and ethanolamine), N-Oleoylethanolamine (the amide of oleic acid (18:1) and ethanolamine), N-Stearoylethanolamine (the amide of stearic acid (18:0) and ethanolamine) and N-Docosahexaenoylethanolamine (the amide of docosahexaenoic acid (22:6) and ethanolamine).

Palmitoylethanolamide (PEA, also known as N-(2-hydroxyethyl) hexadecanamide; Hydroxyethylpalmitamide; palmidrol; N-palmitoylethanolamine; and palmitylethanolamide) is an endogenous fatty acid amide, belonging to the class of nuclear factor agonists. PEA has been demonstrated to bind to a receptor in the cell nucleus (a nuclear receptor) and exerts a variety of biological functions related to chronic pain and inflammation. Studies have shown that PEA interacts with distinct non-CB1/CB2 receptors, suggesting that PEA utilizes a unique “parallel” endocannabinoid signaling system. This concept was further supported by growing evidence that PEA production and inactivation can occur independently of AEA and 2-AG production and inactivation. Much of the biological effects of PEA on cells can be attributed to its affinity to PPAR (particularly PPAR-.alpha. and PPAR-.gamma.). PEA was shown to have an affinity to cannabinoid-like G-coupled receptors GPR55 and GPR119 as well as the transient receptor potential vanilloid type 1 receptor (TRPV1). PEA has been shown to have anti-inflammatory, anti-nociceptive, neuro-protective, and anti-convulsant properties.

### SUMMARY OF THE DISCLOSURE

In some embodiments, there is provided a method for treating cocaine addiction comprising administering to a subject in need thereof a therapeutically acceptable amount of a pharmaceutical composition comprising 5-methoxy-2-aminoindan or a pharmaceutically acceptable salt thereof, thereby treating the cocaine addiction.

In certain embodiments, the 5-methoxy-2-aminoindan or pharmaceutically acceptable salt thereof is administered as a daily dose of about 25 to about 100 mg. In other embodiments, the 5-methoxy-2-aminoindan or pharmaceutically acceptable salt thereof is administered as a daily dose of about 21 to about 84 mg. In still further embodiments, the 5-methoxy-2-aminoindan or pharmaceutically acceptable salt thereof is administered as a daily dose of about 21 to about 100 mg, about 25 to about 90 mg, about 30 to about 80 mg, about 40 to about 70 mg, or about 50 to about 60 mg.

In some embodiments, the daily dose is administered in a single dose or as more than one divided dose. In other embodiments, the 5-methoxy-2-aminoindan or a pharmaceutically acceptable salt thereof is administered twice a day.

In other embodiments, the therapeutically effective amount of 5-methoxy-2-aminoindan or a pharmaceutically acceptable salt thereof comprises about 0.36 to about 1.4 mg/kg body weight/day, about 0.5 to about 1.3 mg/kg body weight/day, about 0.6 to about 1.2 mg/kg body weight/day, about 0.7 to about 1.1 mg/kg body weight/day, or about 0.8 to about 1.0 mg/kg body weight/day.

In certain embodiments, the cocaine addiction is a withdrawal symptom after withdrawal of cocaine.

In some embodiments, the pharmaceutical composition further comprises at least one pharmaceutically acceptable carrier and/or excipient. In particular embodiments, the pharmaceutical composition is a free-flowing powder, a tablet, a capsule, a lozenge, a liquid, a liquid concentrate, suspension, or a syrup.

In other embodiments, the pharmaceutical composition is a unit dosage form composition. In certain embodiments, the amount of 5-methoxy-2-aminoindan or pharmaceutically acceptable salt thereof in the unit dosage form is about 21 to about 100 mg, about 25 to about 90 mg, about 30 to about 80 mg, about 40 to about 70 mg, or about 50 to about 60 mg. In particular embodiments, the amount of 5-methoxy-2-aminoindan or pharmaceutically acceptable salt thereof is about 50 mg.

In some embodiments, administration of the pharmaceutical composition is oral, sublingual, buccal, vaginal, rectal, parenteral, transdermal, or by inhalation. In certain embodiments, the parenteral administration is intravenous, intramuscular, or subcutaneous.

In some embodiments, treating the cocaine addiction attenuates craving for cocaine.

Other embodiments are directed to the use of a pharmaceutical composition comprising 5-methoxy-2-aminoindan or a pharmaceutically acceptable salt thereof for treating cocaine addiction according to any of the preceding embodiments.

In some embodiments, there is provided a method for reducing the likelihood of a relapse of cocaine addiction comprising administering to a subject in need thereof a therapeutically acceptable amount of a pharmaceutical composition comprising 5-methoxy-2-aminoindan or a pharmaceutically acceptable salt thereof, thereby reducing the likelihood of a relapse of cocaine addiction.

In certain embodiments, the 5-methoxy-2-aminoindan or pharmaceutically acceptable salt thereof is administered as a daily dose of about 25 to about 100 mg. In other embodiments, the 5-methoxy-2-aminoindan or pharmaceutically acceptable salt thereof is administered as a daily dose of about 21 to about 84 mg. In still further embodiments, the 5-methoxy-2-aminoindan or pharmaceutically acceptable salt thereof is administered as a daily dose of about 21 to about 100 mg, about 25 to about 90 mg, about 30 to about 80 mg, about 40 to about 70 mg, or about 50 to about 60 mg.

In some embodiments, the daily dose is administered in a single dose or as more than one divided dose. In other embodiments, the 5-methoxy-2-aminoindan or a pharmaceutically acceptable salt thereof is administered twice a day.

In other embodiments, the therapeutically effective amount of 5-methoxy-2-aminoindan or a pharmaceutically acceptable salt thereof comprises about 0.36 to about 1.4 mg/kg body weight/day, about 0.5 to about 1.3 mg/kg body weight/day, about 0.6 to about 1.2 mg/kg body weight/day, about 0.7 to about 1.1 mg/kg body weight/day, or about 0.8 to about 1.0 mg/kg body weight/day.

In certain embodiments, the cocaine addiction is a withdrawal symptom after withdrawal of cocaine.

In some embodiments, the pharmaceutical composition further comprises at least one pharmaceutically acceptable carrier and/or excipient. In particular embodiments, the pharmaceutical composition is a free-flowing powder, a tablet, a capsule, a lozenge, a liquid, a liquid concentrate, suspension, or a syrup.

In other embodiments, the pharmaceutical composition is a unit dosage form composition. In certain embodiments, the amount of 5-methoxy-2-aminoindan or pharmaceutically acceptable salt thereof in the unit dosage form is about 21 to about 100 mg, about 25 to about 90 mg, about 30 to about 80 mg, about 40 to about 70 mg, or about 50 to about 60 mg. In particular embodiments, the amount of 5-methoxy-2-aminoindan or pharmaceutically acceptable salt thereof is about 50 mg.

In some embodiments, administration of the pharmaceutical composition is oral, sublingual, buccal, vaginal, rectal, parenteral, transdermal, or by inhalation. In certain embodiments, the parenteral administration is intravenous, intramuscular, or subcutaneous.

Other embodiments are directed to the use of a pharmaceutical composition comprising 5-methoxy-2-aminoindan or a pharmaceutically acceptable salt thereof for reducing the likelihood of a relapse of cocaine addiction according to claims 18-32.

In some embodiments, there is provided a method for treating cocaine addiction comprising administering to a subject in need thereof a therapeutically acceptable amount of a pharmaceutical composition comprising 5-methoxy-2-aminoindan or a pharmaceutically acceptable salt thereof, and an N-acylethanolamine or a pharmaceutically acceptable salt thereof, thereby treating the cocaine addiction.

In certain embodiments, the 5-methoxy-2-aminoindan or pharmaceutically acceptable salt thereof is administered as a daily dose of about 25 to about 100 mg. In other embodiments, the 5-methoxy-2-aminoindan or pharmaceutically acceptable salt thereof is administered as a daily dose of about 21 to about 84 mg. In still further embodiments, the 5-methoxy-2-aminoindan or pharmaceutically acceptable salt thereof is administered as a daily dose of about 21 to about 100 mg, about 25 to about 90 mg, about 30 to about 80 mg, about 40 to about 70 mg, or about 50 to about 60 mg.

In some embodiments, the daily dose is administered in a single dose or as more than one divided dose. In other embodiments, the 5-methoxy-2-aminoindan or a pharmaceutically acceptable salt thereof is administered twice a day.

In other embodiments, the therapeutically effective amount of 5-methoxy-2-aminoindan or a pharmaceutically acceptable salt thereof comprises about 0.36 to about 1.4 mg/kg body weight/day, about 0.5 to about 1.3 mg/kg body weight/day, about 0.6 to about 1.2 mg/kg body weight/day, about 0.7 to about 1.1 mg/kg body weight/day, or about 0.8 to about 1.0 mg/kg body weight/day.

In certain embodiments, the cocaine addiction is a withdrawal symptom after withdrawal of cocaine.

In some embodiments, the pharmaceutical composition further comprises at least one pharmaceutically acceptable carrier and/or excipient. In particular embodiments, the pharmaceutical composition is a free-flowing powder, a tablet, a capsule, a lozenge, a liquid, a liquid concentrate, suspension, or a syrup.

In other embodiments, the pharmaceutical composition is a unit dosage form composition. In certain embodiments, the amount of 5-methoxy-2-aminoindan or pharmaceutically acceptable salt thereof in the unit dosage form is about 21 to about 100 mg, about 25 to about 90 mg, about 30 to about 80 mg, about 40 to about 70 mg, or about 50 to about 60 mg. In particular embodiments, the amount of 5-methoxy-2-aminoindan or pharmaceutically acceptable salt thereof is about 50 mg.

In some embodiments, administration of the pharmaceutical composition is oral, sublingual, buccal, vaginal, rectal, parenteral, transdermal, or by inhalation. In certain embodiments, the parenteral administration is intravenous, intramuscular, or subcutaneous.

In some embodiments, the N-acylethanolamine is palmitoylethanolamide or a pharmaceutically acceptable salt thereof. In particular embodiments, the N-acylethanolamine or pharmaceutically acceptable salt thereof is administered as a daily dose of about 200 to about 1800 mg, about 250 to about 1550 mg, about 300 to about 1200 mg, about 350 to about 950 mg, about 400 to about 700 mg, about 450 to about 600 mg, or about 500 to about 550 mg.

In some embodiments, treating the cocaine addiction attenuates craving for cocaine.

Other embodiments are directed to the use of a pharmaceutical composition comprising 5-methoxy-2-aminoindan or a pharmaceutically acceptable salt thereof, and palmitoylethanolamide or a pharmaceutically acceptable salt thereof, for treating cocaine addiction according to any of the preceding embodiments.

In some embodiments, there is provided a method for reducing the likelihood of a relapse of cocaine addiction comprising administering to a subject in need thereof a therapeutically acceptable amount of a pharmaceutical composition comprising 5-methoxy-2-aminoindan or a pharmaceutically acceptable salt thereof, and an N-acylethanolamine or a pharmaceutically acceptable salt thereof, thereby reducing the likelihood of a relapse of cocaine addiction.

In certain embodiments, the 5-methoxy-2-aminoindan or pharmaceutically acceptable salt thereof is administered as a daily dose of about 25 to about 100 mg. In other embodiments, the 5-methoxy-2-aminoindan or pharmaceutically acceptable salt thereof is administered as a daily dose of about 21 to about 84 mg. In still further embodiments, the 5-methoxy-2-aminoindan or pharmaceutically acceptable salt thereof is administered as a daily dose of about 21 to about 100 mg, about 25 to about 90 mg, about 30 to about 80 mg, about 40 to about 70 mg, or about 50 to about 60 mg.

In some embodiments, the daily dose is administered in a single dose or as more than one divided dose. In other embodiments, the 5-methoxy-2-aminoindan or a pharmaceutically acceptable salt thereof is administered twice a day.

In other embodiments, the therapeutically effective amount of 5-methoxy-2-aminoindan or a pharmaceutically acceptable salt thereof comprises about 0.36 to about 1.4 mg/kg body weight/day, about 0.5 to about 1.3 mg/kg body weight/day, about 0.6 to about 1.2 mg/kg body weight/day, about 0.7 to about 1.1 mg/kg body weight/day, or about 0.8 to about 1.0 mg/kg body weight/day.

In certain embodiments, the cocaine addiction is a withdrawal symptom after withdrawal of cocaine.

In some embodiments, the pharmaceutical composition further comprises at least one pharmaceutically acceptable carrier and/or excipient. In particular embodiments, the pharmaceutical composition is a free-flowing powder, a tablet, a capsule, a lozenge, a liquid, a liquid concentrate, suspension, or a syrup.

In other embodiments, the pharmaceutical composition is a unit dosage form composition. In certain embodiments, the amount of 5-methoxy-2-aminoindan or pharmaceutically acceptable salt thereof in the unit dosage form is about 21 to about 100 mg, about 25 to about 90 mg, about 30 to about 80 mg, about 40 to about 70 mg, or about 50 to about 60 mg. In particular embodiments, the amount of 5-methoxy-2-aminoindan or pharmaceutically acceptable salt thereof is about 50 mg.

In some embodiments, administration of the pharmaceutical composition is oral, sublingual, buccal, vaginal, rectal, parenteral, transdermal, or by inhalation. In certain embodiments, the parenteral administration is intravenous, intramuscular, or subcutaneous.

In some embodiments, the N-acylethanolamine is palmitoylethanolamide or a pharmaceutically acceptable salt thereof. In particular embodiments, the N-acylethanolamine or pharmaceutically acceptable salt thereof is administered as a daily dose of about 200 to about 1800 mg, about 250 to about 1550 mg, about 300 to about 1200 mg, about 350 to about 950 mg, about 400 to about 700 mg, about 450 to about 600 mg, or about 500 to about 550 mg.

Other embodiments are directed to the use of a pharmaceutical composition comprising 5-methoxy-2-aminoindan or a pharmaceutically acceptable salt thereof, and an N-acylethanolamine or a pharmaceutically acceptable salt thereof, for reducing the likelihood of a relapse of cocaine addiction according to claims 53-69.

### BRIEF DESCRIPTION OF THE FIGURES

The foregoing summary, as well as the following detailed description of the disclosure, will be better understood when read in conjunction with the appended drawings. For the purpose of illustrating the present disclosure, the attached drawings illustrate some, but not all, alternative embodiments. It should be understood, however, that the disclosure is not limited to the precise arrangements and instrumentalities shown. These figures, which are incorporated into and constitute part of the specification, assist in explaining the principles of the disclosures.

FIG. 1 shows data from a Conditioned Place Preference (“CPP”) assay, wherein the Y-axis represents time spent in the compartment that was less preferred during baseline testing. The behavioral data were analyzed with ordinary one-way ANOVA. Data was expressed as mean±SEM and a probability value of ****p<0.0001 was considered significant.

FIG. 2 shows data from a CPP assay, wherein the Y-axis represents time spent in the compartment that was less preferred during baseline testing. The behavioral data were analyzed with ordinary one-way ANOVA. Data was expressed as means±SEM and a probability value of *p<0.001 was considered significant. The K-Cluster method was used to partition the MEAI (5 mg/kg) values into two distinct groups. MEAI A represents values below 100 (sec) and MEAI B represents values above 100 (sec).

### DETAILED DESCRIPTION OF THE DISCLOSURE

The present invention, in some embodiments, relates to cocaine addiction, and more particularly, but not exclusively, to methods for treating cocaine addiction comprising administering to a subject in need thereof a therapeutically effective amount of MEAI, or a pharmaceutically acceptable salt thereof. In other embodiments, the disclosure provides methods for reducing the likelihood of a relapse of cocaine addiction comprising administering to a subject in need thereof a therapeutically acceptable amount of a pharmaceutical composition comprising 5-methoxy-2-aminoindan or a pharmaceutically acceptable salt thereof.

The disclosure also provides methods for treating cocaine addiction comprising administering to a subject in need thereof a therapeutically effective amount of MEAI, or a physiologically acceptable salt thereof, and an N-acylethanolamine, or a pharmaceutically acceptable salt thereof. In other embodiments, the disclosure provides methods for reducing the likelihood of a relapse of cocaine addiction comprising administering to a subject in need thereof a therapeutically acceptable amount of a pharmaceutical composition comprising 5-methoxy-2-aminoindan or a pharmaceutically acceptable salt thereof, and an N-acylethanolamine, or a physiologically acceptable salt thereof. In some embodiments, the N-acylethanolamine is PEA.

### Definitions

“Isomers” means compounds having the same number and kind of atoms, and hence the same molecular weight, but differing with respect to the arrangement or configuration of the atoms in space.

“Stereoisomer” or “optical isomer” mean a stable isomer that has at least one chiral atom or restricted rotation giving rise to perpendicular dissymmetric planes (e.g., certain biphenyls, allenes, and spiro compounds) and can rotate plane-polarized light. Because asymmetric centers and other chemical structure exist in the compounds of the disclosure which may give rise to stereoisomerism, the disclosure contemplates stereoisomers and mixtures thereof. The compounds of the disclosure and their salts include asymmetric carbon atoms and may therefore exist as single stereoisomers, racemates, and as mixtures of enantiomers and diastereomers. Typically, such compounds will be prepared as a racemic mixture. If desired, however, such compounds can be prepared or isolated as pure stereoisomers, i.e., as individual enantiomers or diastereomers, or as stereoisomer-enriched mixtures. As discussed in more detail below, individual stereoisomers of compounds are prepared by synthesis from optically active starting materials containing the desired chiral centers or by preparation of mixtures of enantiomeric products followed by separation or resolution, such as conversion to a mixture of diastereomers followed by separation or recrystallization, chromatographic techniques, use of chiral resolving agents, or direct separation of the enantiomers on chiral chromatographic columns. Starting compounds of particular stereochemistry are either commercially available or are made by the methods described below and resolved by techniques well-known in the art.

It is well-known in the art that the biological and pharmacological activity of a compound is sensitive to the stereochemistry of the compound. Thus, for example, enantiomers often exhibit strikingly different biological activity including differences in pharmacokinetic properties, including metabolism, protein binding, and the like, and pharmacological properties, including the type of activity displayed, the degree of activity, toxicity, and the like. Thus, one skilled in the art will appreciate that one enantiomer may be more active or may exhibit beneficial effects when enriched relative to the other enantiomer or when separated from the other enantiomer. Additionally, one skilled in the art would know how to separate, enrich, or selectively prepare the enantiomers of the compounds of the disclosure from this disclosure and the knowledge of the prior art.

Thus, although the racemic form of drug may be used, it is often less effective than administering an equal amount of enantiomerically pure drug; indeed, in some cases, one enantiomer may be pharmacologically inactive and would merely serve as a simple diluent. For example, although ibuprofen had been previously administered as a racemate, it has been shown that only the S-isomer of ibuprofen is effective as an anti-inflammatory agent (in the case of ibuprofen, however, although the R-isomer is inactive, it is converted in vivo to the S-isomer, thus, the rapidity of action of the racemic form of the drug is less than that of the pure S-isomer). Furthermore, the pharmacological activities of enantiomers may have distinct biological activity. For example, S-penicillamine is a therapeutic agent for chronic arthritis, while R-penicillamine is toxic. Indeed, some purified enantiomers have advantages over the racemates, as it has been reported that purified individual isomers have faster transdermal penetration rates compared to the racemic mixture. See U.S. Pat. Nos. 5,114,946 and 4,818,541.

In some embodiments, the compound is a racemic mixture of (S)- and (R)-isomers. In other embodiments, provided herein is a mixture of compounds wherein individual compounds of the mixture exist predominately in an (S)- or (R)-isomeric configuration. For example, the compound mixture has an (S)-enantiomeric excess of greater than about 55%, about 60%, about 65%, about 70%, about 75%, about 80%, about 85%, about 90%, about 95%, about 96%, about 97%, about 98%, about 99%, about 99.5%, or more. In other embodiments, the compound mixture has an (S)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5%, or more. In other embodiments, the compound mixture has an (R)-enantiomeric purity of greater than about 55%, about 60%, about 65%, about 70%, about 75%, about 80%, about 85%, about 90%, about 95%, about 96%, about 97%, about 98%, about 99%, about 99.5% or more. In some other embodiments, the compound mixture has an (R)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5% or more.

Individual stereoisomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by: (1) attachment of a mixture of enantiomers to a chiral auxiliary, separation of the resulting mixture of diastereomers by recrystallization or chromatography and liberation of the optically pure product from the auxiliary; (2) salt formation employing an optically active resolving agent; or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well-known asymmetric synthetic methods.

Thus, if one enantiomer is pharmacologically more active, less toxic, or has a preferred disposition in the body than the other enantiomer, it would be therapeutically more beneficial to administer that enantiomer preferentially. In this way, the patient undergoing treatment would be exposed to a lower total dose of the drug and to a lower dose of an enantiomer that is possibly toxic or an inhibitor of the other enantiomer.

As used herein, nomenclature for compounds including organic compounds, can be given using common names, IUPAC, IUBMB, or CAS recommendations for nomenclature. One of skill in the art can readily ascertain the structure of a compound if given a name, either by systemic reduction of compound structure using naming conventions, or by commercially available software, such as CHEMDRAW™ (Cambridgesoft Corporation, U.S.A.). Chemical names were generated using PerkinElmer ChemDraw® Professional, version 17.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbol “R” or “S,” depending on the configuration of substituents around the stereogenic carbon atom. The present disclosure encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. In some embodiments, an enantiomer or stereoisomer may be provided substantially free of the corresponding enantiomer.

The present invention provides, in one aspect, a pharmaceutical composition comprising a therapeutically-effective amount of a mixture of MEAI or a salt thereof and at least one N-acylethanolamine or a salt thereof.

The present invention provides, in another aspect, a pharmaceutical composition comprising a therapeutically-effective amount of a mixture of MEAI or a salt thereof and at least one N-acylethanolamine or a salt thereof, wherein the molar ratio between the MEAI and the N-acylethanolamine is between about 1:0.2 to about 1:2000.

As used herein, a “pharmaceutical composition” refers to a preparation of the active agents described herein with other chemical components such as physiologically suitable carriers and excipients. The purpose of a pharmaceutical composition is to facilitate administration of a compound to an organism. As used herein, the phrase “pharmaceutically acceptable carrier” refers to a carrier, an excipient or a diluent that does not cause significant irritation to an organism and does not abrogate the biological activity and properties of the administered compound. An adjuvant is included under these phrases.

The term “excipient” as used herein refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, oils such as vegetable oils or fish oils, and polyethylene glycols.

The term “carrier” as used herein refers to a diluent, adjuvant, excipient, or vehicle with which the compound is administered. Such pharmaceutical carriers can be sterile liquids, such as water and oils. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition.

The phrase “pharmaceutically acceptable” as used herein refers to molecular entities and compositions that are physiologically tolerable and do not typically produce an allergic or similar toxicity when administered to an individual. Preferably, and particularly where a formulation is used in humans, the term “pharmaceutically acceptable” may mean approved by a regulatory agency (for example, the U.S. Food and Drug Agency) or listed in a generally recognized pharmacopeia for use in animals (e.g., the U.S. Pharmacopeia).

The term “N-acylethanolamine” as used herein generally refers to a type of fatty acid amide, lipid-derived signaling molecules, formed when one of several types of acyl group is linked to the nitrogen atom of ethanolamine. These amides conceptually can be formed from a fatty acid and ethanolamine with the release of a molecule of water, but the known biological synthesis uses a specific phospholipase D to cleave the phospholipid unit from N-acylphosphatidylethanolamines. The suffixes -amine and -amide in these names each refer to the single nitrogen atom of ethanolamine that links the compound together: it is termed “amine” in ethanolamine because it is considered as a free terminal nitrogen in that subunit, while it is termed “amide” when it is considered in association with the adjacent carbonyl group of the acyl subunit. Names for these compounds may be encountered with either “amide” or “amine” in the present application. The term “ethanolamine” is used in the generic sense and is meant to include mono-ethanolamine, di-ethanolamine, tri-ethanolamine, and mixtures thereof.

The term “derivative” as used herein means a compound whose core structure is the same as, or closely resembles that of an N-acylethanolamine compound, but which has a chemical or physical modification, such as different or additional side groups.

The term “salt” as used herein refers to any form of an active ingredient in which the active ingredient assumes an ionic form and is coupled to a counter ion (a cation or anion) or is in solution. This also includes complexes of the active ingredient with other molecules and ions, in particular complexes which are complexed by ion interaction. Pharmaceutically acceptable salts are known to persons of ordinary skill in the art.

In certain embodiments, the molar ratio between the MEAI and the N-acylethanolamine is between about 1:0.2 to about 1:5. In certain embodiments, the molar ratio between the MEAI and the N-acylethanolamine is between about 1:0.22 to about 1:4.5, about 1:0.25 to about 1:4, between about 1:0.28 to about 1:3.5, between about 1:0.33 to about 1:3, between about 1:0.4 to about 1:2.5, between about 1:0.5 to about 1:2 or about 1:1. Each possibility represents a separate embodiment of the present invention.

In certain embodiments, the molar ratio between the MEAI and the N-acylethanolamine is between about 1:15 to about 1:1800. In certain embodiments, the molar ratio between the MEAI and the N-acylethanolamine is between about 1:16 to about 1:1700, about 1:17 to about 1:1600, about 1:18 to about 1:1500, about 1:19 to about 1:1400, about 1:20 to about 1:1300, about 1:21 to about 1:1200, about 1:22 to about 1:1100, about 1:23 to about 1:1000, about 1:24 to about 1:900, about 1:15 to about 1:800, about 1:16 to about 1:700, about 1:17 to about 1:600, about 1:18 to about 1:500, about 1:19 to about 1:490, about 1:20 to about 1:480, about 1:21 to about 1:470, or about 1:22 to about 1:460. Each possibility represents a separate embodiment of the present invention. In certain embodiments, the molar ratio between the MEAI and the N-acylethanolamine is between about 1:25 to about 1:450. In certain embodiments, the molar ratio between the MEAI and the N-acylethanolamine is between about 1:10 to about 1:500, about 1:15 to about 1:450, about 1:20 to about 1:400, about 1:25 to about 1:350, about 1:30 to about 1:300, about 1:35 to about 1:250, about 1:40 to about 1:200, or about 1:45 to about 1:150. Each possibility represents a separate embodiment of the present invention. In certain embodiments, the molar ratio between the MEAI and the N-acylethanolamine is between about 1:50 to about 1:100. In certain embodiments, the molar ratio between the MEAI and the N-acylethanolamine is about 1:10. In certain embodiments, the molar ratio between the MEAI and the N-acylethanolamine is about 1:20. In certain embodiments, the molar ratio between the MEAI and the N-acylethanolamine is about 1:30. In certain embodiments, the molar ratio between the MEAI and the N-acylethanolamine is about 1:40. In certain embodiments, the molar ratio between the MEAI and the N-acylethanolamine is about 1:50. In certain embodiments, the molar ratio between the MEAI and the N-acylethanolamine is about 1:60. In certain embodiments, the molar ratio between the MEAI and the N-acylethanolamine is about 1:70. In certain embodiments, the molar ratio between the MEAI and the N-acylethanolamine is about 1:80. In certain embodiments, the molar ratio between the MEAI and the N-acylethanolamine is about 1:90. In certain embodiments, the molar ratio between the MEAI and the N-acylethanolamine is about 1:100. In certain embodiments, the molar ratio between the MEAI and the N-acylethanolamine is about 1:110. In certain embodiments, the molar ratio between the MEAI and the N-acylethanolamine is about 1:120. In certain embodiments, the molar ratio between the MEAI and the N-acylethanolamine is about 1:130. In certain embodiments, the molar ratio between the MEAI and the N-acylethanolamine is about 1:140. In certain embodiments, the molar ratio between the MEAI and the N-acylethanolamine is about 1:150. In certain embodiments, the molar ratio between the MEAI and the N-acylethanolamine is about 1:160. In certain embodiments, the molar ratio between the MEAI and the N-acylethanolamine is about 1:170. In certain embodiments, the molar ratio between the MEAI and the N-acylethanolamine is about 1:180. In certain embodiments, the molar ratio between the MEAI and the N-acylethanolamine is about 1:190. In certain embodiments, the molar ratio between the MEAI and the N-acylethanolamine is about 1:200. In certain embodiments, the molar ratio between the MEAI and the N-acylethanolamine is at least about 1:10, at least about 1:20, at least about 1:30, at least about 1:40, at least about 1:50, at least about 1:60, at least about 1:70, at least about 1:80, at least about 1:90, or at least about 1:100. Each possibility represents a separate embodiment of the present invention.

In certain embodiments, the pharmaceutical composition comprises about 0.5-10 mg MEAI or a salt thereof. In certain embodiments, the pharmaceutical composition comprises about 1-9.5 mg, about 1.5-9 mg, about 2-8.5 mg, about 2.5-8 mg, about 3-7.5 mg, about 3.5-7 mg, about 4-6.5 mg, about 4.5-6 mg or about 5-5.5 mg MEAI or a salt thereof. In certain embodiments, the pharmaceutical composition comprises about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg or about 10 mg MEAI or a salt thereof. Each possibility represents a separate embodiment of the present invention. In certain embodiments, the pharmaceutical composition comprises less than about 0.5 mg, less than about 1 mg, less than about 1.5 mg, less than about 2 mg, less than about 2.5 mg, less than about 3 mg, less than about 3.5 mg, less than about 4 mg, less than about 4.5 mg, less than about 5 mg, less than about 5.5 mg, less than about 6 mg, less than about 6.5 mg, less than about 7 mg, less than about 7.5 mg, less than about 8 mg, less than about 8.5 mg, less than about 9 mg, less than about 9.5 mg or about 10 mg MEAI or a salt thereof. Each possibility represents a separate embodiment of the present invention. In certain embodiments, the pharmaceutical composition comprises about 0.5 mg to about 1 mg, about 0.5 mg to about 1.5 mg, about 0.5 mg to about 2 mg, about 0.5 mg to about 2.5 mg, about 0.5 mg to about 3 mg, about 0.5 mg to about 3.5 mg, about 0.5 mg to about 4 mg, about 0.5 mg to about 4.5 mg, about 0.5 mg to about 5 mg, about 0.5 mg to about 5.5 mg, about 0.5 mg to about 6 mg, about 0.5 mg to about 6.5 mg, about 0.5 mg to about 7 mg, about 0.5 mg to about 7.5 mg, about 0.5 mg to about 8 mg, about 0.5 mg to about 8.5 mg, about 0.5 mg to about 9 mg or about 0.5 mg to about 9.5 mg MEAI or a salt thereof. Each possibility represents a separate embodiment of the present invention.

In certain embodiments, the pharmaceutical composition comprises about 200-1800 mg N-acylethanolamine or a salt thereof. In certain embodiments, the pharmaceutical composition comprises about 250-1550 mg, about 300-1200 mg, about 350-950 mg, about 400-700 mg, about 450-600 mg or about 500-550 mg N-acylethanolamine or a salt thereof. Each possibility represents a separate embodiment of the present invention. In certain embodiments, the pharmaceutical composition comprises at least about 50 mg, at least about 100 mg, at least about 150 mg, at least about 200 mg, at least about 250 mg, at least about 300 mg, at least about 350 mg, at least about 400, at least about 450 mg, at least about 500 mg, at least about 550 mg, at least about 600 mg, at least about 650 mg, at least about 700 mg, at least about 750 mg, at least about 800 mg, at least about 850 mg, at least about 900 mg, at least about 950 mg, at least about 1000 mg, at least about 1050 mg, at least about 1100 mg, at least about 1150 mg, at least about 1200 mg, at least about 1250 mg, at least about 1300 mg, at least about 1350 mg, at least about 1400 mg, at least about 1450 mg, at least about 1500 mg, at least about 1550 mg, at least about 1600 mg, at least about 1650 mg, at least about 1700 mg, at least about 1750 mg or at least about 1800 mg N-acylethanolamine or a salt thereof. In certain embodiments, the pharmaceutical composition comprises about 50 mg, about 100 mg, about 150 mg, about 200 mg, about 250 mg, about 300 mg, about 350 mg, about 400, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg or about 1800 mg N-acylethanolamine or a salt thereof. Each possibility represents a separate embodiment of the present invention.

In certain embodiments, the N-acylcthanolamine is N-palmitoylethanolamine (PEA), Me-palmitoylethanolamide (Me-PEA), palmitoylcyclohexamide, palmitoylbutylamide, palmitoylisopropylamide, oleoylethanolamine (OEA), palmitoylisopropylamide (PIA), or salts thereof, or any combination thereof. Each possibility represents a separate embodiment of the present invention. In certain embodiments, the N-acylethanolamine is PEA or a salt thereof. In certain embodiments, the N-acylethanolamine consists of PEA or a salt thereof. In certain embodiments, the N-acylethanolamine consists of PEA.

In certain embodiments, the pharmaceutical composition is formulated for systemic administration. In certain embodiments, the pharmaceutical composition is formulated for oral, oral mucosal, nasal, sublingual, inhalational, topical, rectal, vaginal, parenteral, intravenous, intramuscular, or subcutaneous administration. In certain embodiments, the pharmaceutical composition is formulated for oral, oral mucosal, nasal, or sublingual administration. Each possibility represents a separate embodiment of the present invention. In certain embodiments, the pharmaceutical composition is formulated for oral administration. In certain embodiments, the pharmaceutical composition is formulated for oral mucosal administration. In certain embodiments, the pharmaceutical composition is formulated for nasal administration. In certain embodiments, the pharmaceutical composition is formulated for sublingual administration.

Techniques for formulation and administration of drugs are well known in the art, and may be found, e.g. in “Remington's Pharmaceutical Sciences,” Mack Publishing Co., Easton, Pa. Pharmaceutical compositions of the present invention may be manufactured by processes well known in the art, e.g., by means of conventional mixing, dissolving, granulating, dragee-making, levigating, emulsifying, encapsulating, entrapping, or lyophilizing processes.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries as desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, and sodium carbomethylcellulose; and/or physiologically acceptable polymers such as polyvinylpyrrolidone (PVP). If desired, disintegrating agents, such as cross-linked polyvinyl pyrrolidone, agar, or alginic acid or a salt thereof, such as sodium alginate, may be added.

The term “oral administration” refers to any method of administration in which an active agent can be administered by swallowing, chewing, sucking, or drinking an oral dosage form. Examples of solid dosage forms include conventional tablets, multi-layer tablets, capsules, caplets, etc., which do not substantially release the drug in the mouth or in the oral cavity.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions that can be used orally include stiff or soft, sealed capsules made of gelatin and a plasticizer, such as glycerol or sorbitol. The capsules may contain the active ingredients in admixture with filler such as lactose, binders such as starches, lubricants such as talc or magnesium stearate, and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration. For buccal and sublingual administration, the compositions may take the form of tablets or lozenges formulated in conventional manner or in adhesive carriers. Alternatively, the active ingredient may be in powder form for constitution with a suitable vehicle, e.g., a sterile, pyrogen-free, water-based solution, before use.

Pharmaceutical compositions suitable for use in the context of the present invention include compositions wherein the active ingredients are contained in an amount effective to achieve the intended purpose. More specifically, a “therapeutically effective amount” means an amount of active ingredients effective to prevent, alleviate, or ameliorate symptoms or side effects of a disease or disorder, or prolong the survival of the subject being treated. Determination of a therapeutically effective amount is well within the capability of those skilled in the art, especially in light of the detailed disclosure provided herein. More specifically, a “therapeutically effective amount of a mixture” means an amount of at least two active ingredients, wherein each one of the active ingredients independently may not be in a therapeutically effective amount or wherein both of the active ingredients may not be in a therapeutically effective amount, the mixture is nevertheless effective to prevent, alleviate, or ameliorate symptoms or side effects of a disease or disorder, or prolong the survival of the subject being treated. The term “mixture” as used herein refers to a non-covalent combination of two molecules.

For any preparation used in the methods of the invention, the dosage or the therapeutically effective amount can be estimated initially from in vitro, in vivo and cell culture assays. For example, a dose can be formulated in animal models to achieve a desired concentration or titer. Such information can be used to more accurately determine useful doses in humans. The dosage of each compound of the claimed combinations depends on several factors, including: the administration method, the disease to be treated, the severity of the disease, whether the disease is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect dosage used. Continuous daily dosing may not be required; a therapeutic regimen may require cycles, during which time a drug is not administered, or therapy may be provided on an as-needed basis during periods of acute disease worsening. Dosage escalation may or may not be required; a therapeutic regimen may require reduction in medication dosage. Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration, and dosage can be chosen by the individual physician in view of the patient's condition (See, e.g., Fingl, E. et al. (1975), “The Pharmacological Basis of Therapeutics,” Ch. 1, p. 1). Depending on the severity and responsiveness of the condition to be treated, dosing can be of a single or a plurality of administrations, with course of treatment lasting from several days to several weeks, or until cure is effected or diminution of the disease state is achieved.

The present invention further provides, in another aspect, a dosage unit comprising or consisting of the pharmaceutical composition described above.

In certain embodiments, the dosage unit comprises the pharmaceutical composition described above. In certain embodiments, the dosage unit consisting of the pharmaceutical composition described above. In certain embodiments, the dosage unit is formulated as a gel, a powder or a spray. In certain embodiments, the dosage unit is formulated as a gel. In certain embodiments, the dosage unit is formulated as a powder. In certain embodiments, the dosage unit is formulated as a spray.

The present invention further provides, in another aspect, a pharmaceutical composition or a dosage unit as described above for use in a method for preventing or treating a condition amenable to prevention or treatment by at least one MEAI.

The term “treating” as used herein, includes, but is not limited to, any one or more of the following: abrogating, ameliorating, inhibiting, attenuating, blocking, suppressing, reducing, delaying, halting, alleviating or preventing one or more symptoms or side effects of the diseases or conditions of the invention.

The term “acute” refers to a condition with a relatively short, severe course.

The term “chronic” as used herein means that the length of time of the diseases or conditions of the invention can be weeks, months, or possibly years. The intensity of the diseases or conditions can differentiate according to various conditions such as patient age, temperature, season, type of disease, etc.

The term “about” as used herein in relation to a value, a plurality of values or a range of values defined by a lowest and highest values means a value which is 10% lower and/or higher than the corresponding value, plurality of values or range of values. For example, the phrase “about 1” means “0.9 to 1.1”, the phrase “about 1 or 2” means “0.9 to 1.1 or 1.8 to 2.2”, and the phrase “about 1 to about 2” means “0.9 to 2.2”.

The terms “comprises”, “comprising”, “includes”, “including”, “having” and their conjugates mean “including but not limited to.”

The term “consisting of” means “including and limited to.”

The term “consisting essentially of” means that the composition, method or microcapsules may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferable.

Data obtained from the cell culture assays or animal studies can be used in formulating a range of dosage for use in humans. Therapeutically effective dosages achieved in one animal model may be converted for use in another animal, including humans, using conversion factors known in the art (see, e.g., Freireich et al., Cancer Chemother. Reports 50(4):219-244 (1966) and the following Table for Equivalent Surface Area Dosage Factors).

TABLE 2
Equivalent Surface Area Dosage Factors.

To:

| | Mouse | Rat | Monkey | Dog | Human |
|---|---|---|---|---|---|
| From: | (20 g) | (150 g) | (3.5 kg) | (8 kg) | (60 kg) |
| Mouse | 1 | ½ | ¼ | ⅙ | 1/12 |
| Rat | 2 | 1 | ½ | ¼ | 1/7 |
| Monkey | 4 | 2 | 1 | ⅗ | ⅓ |
| Dog | 6 | 4 | ⅗ | 1 | ½ |
| Human | 12 | 7 | 3 | 2 | 1 |

The dosage of such compounds lies preferably within a range of circulating concentrations that include the ED₅₀ with little or no toxicity. The dosage may vary within this range depending upon the dosage form employed and the route of administration utilized. Generally, a therapeutically effective amount may vary with the subject's age, condition, and gender, as well as the severity of the medical condition in the subject. The dosage may be determined by a physician and adjusted, as necessary, to suit observed effects of the treatment.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trials including first-in-human, dose ranging and efficacy trials, in healthy patients and/or those suffering from a given disorder, may be completed according to methods well known in the clinical and medical arts.

While the present invention has been described with reference to certain embodiments, it will be understood by those skilled in the art that various changes may be made and equivalents may be substituted without departing from the scope of the present invention. In addition, many modifications may be made to adapt a particular situation or material to the teachings of the present invention without departing from its scope. Therefore, it is intended that the present invention is not limited to the particular embodiment disclosed, but that the present invention will include all embodiments falling within the scope of the appended claims.

The following examples are presented to more fully illustrate some embodiments of the invention. They should, in no way be construed, however, as limiting the broad scope of the invention.

### Example 1: Elucidation of MEAI as an Anti-Reward/Addictive-Like Agent

To investigate MEAI's anti-rewarding effect. we looked for a dose that will not be rewarding and addicting using rats in the Conditioned Place Preference (CPP) model. In this model, if a drug is rewarding the rat will demonstrate a preference of spending time in a compartment where it received the drug on each conditioning day. If MEAI is a non-rewarding agent, then one would expect the rat to not demonstrate a preference for the compartment associated with the MEAI.

Two groups were tested: One group received MEAI (four different doses), while the second group received cocaine in each conditioning day. Additionally, some rats that received cocaine were treated with MEAI on the test day to determine if MEAI has a treatment-like effect.

In the CPP assay, the rats learned to associate a reward with an environmental cue. Two groups of rats were put through a three-day baseline period during which each rat was allowed to freely move during 20 minutes from a striped to a smooth compartment in a closed arena. Time measurements were taken to determine which compartment the rat spent more time in (i.e., preferred). Baseline data was determined as an average duration of time spent in each compartment. After the baseline period, a training period of 5 days was conducted in which rats were injected with saline (I.P.) in the morning and placed in the preferred compartment for 20 minutes. Four hours after the saline injection, cocaine (15 mg/kg I.P.) or MEAI (4 doses; 20, 10, 5 and 2.5 mg/kg I.P.) was injected and the rat was placed in the less preferred compartment for 20 minutes. On the 6th day, a CPP test was conducted (identical to the baseline period) to determine if MEAI imparts a reward-like effect similar to cocaine. The duration time spent in each compartment was calculated as the difference between the baseline time and the testing time (seconds) as previously described.

### Elucidation of MEAI as Anti-Reward/Addictive-Like Agent

In the first part of this study, we examined whether administration of MEAI in four different doses had no effect on reward.

We started testing with 20 mg/kg of MEAI. At this dose, 65.7% of the rats presented side effects such as anxiety, aggressiveness, piloerection, tremors, low body posture, diarrhea, and secretion of “vaginal plug.” Rats receiving this dose demonstrated low duration of time spent in a non-preferred place (sec) which indicates a low reward. Due to these side effects, we decided to test lower doses of MEAI as opposed to higher doses. Accordingly, the following dose tested was 10 mg/kg of MEAI. 51% of the rats receiving this dose had side effects, including anxiety, aggressiveness, piloerection, tremors low body posture, diarrhea, and secretion of “vaginal plug,” with a trend towards improved symptoms, although not statistically significant, compared to the 20 mg/kg dose. We saw a higher duration of time spent in a non-preferred place with this dose, indicating that there was a rewarding effect. Based on these results, we chose 5 and 2.5 mg/kg as the next two doses to administer. With the 5 mg/kg dose 20.2% of the rats presented side effects, and with the 2.5 mg/kg dose 21.4% of the rats presented side effects, which consisted of anxiety, diarrhea, and secretion of “vaginal plug.” For these two doses, the duration of time in a non-preferred place was negative, demonstrating an anti-reward effect. Overall, the mean that was most negative was for the dose 5 mg/kg, and therefore we decided to test the efficacy of this dose as a treatment for cocaine preference in rats.

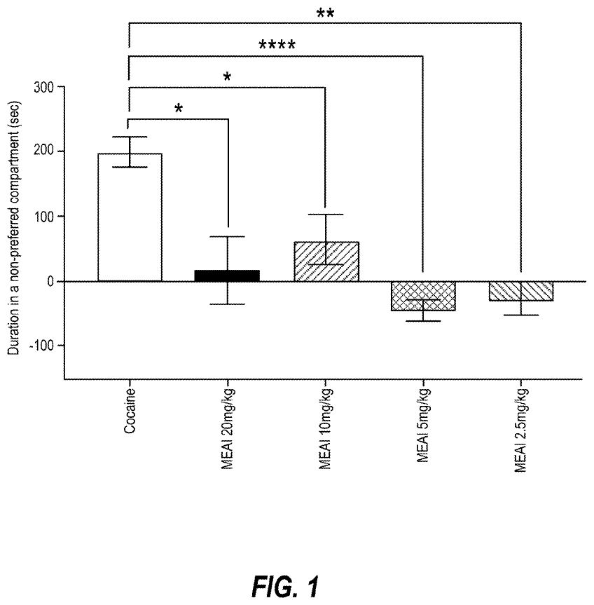

FIG. 1 shows the results of a CPP assay. Y-axis represents time spent in the compartment that was less preferred during baseline testing. The behavioral data were analyzed with Ordinary one-way ANOVA. Data was expressed as mean±SEM and a probability value of ****p<0.0001 was considered significant. n=5-14 rats per group.

### Testing MEAI as a Treatment for Cocaine Preference in Rats.

Based on the previous results, we chose 5 mg/kg MEAI as a treatment dose since it had a satisfying anti-reward effect. 12 rats were conditioned with cocaine (15 mg/kg) and on the day of the test were injected with MEAI (5 mg/kg i.p), followed by a 10-minute wait in their home cage. The treated rats were then inserted into the middle compartment of the arena for the test. The results were mixed, that is, there were rats that showed preference to the non-preferred compartment (negative values) and rats that still preferred the baseline preferred compartment (positive values). We used a K-cluster test to determine if within the group we could separate two different groups with statistical significance.

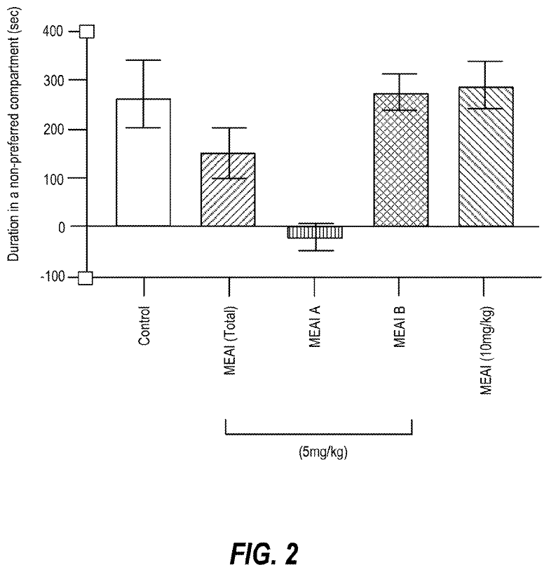

The two groups were divided as values below 100 seconds in a non-preferred compartment (MEAI A) and above 100 seconds (MEAI B). We then decided to examine a higher dose (10 mg/kg i.p) to see if a higher dose was a more effective treatment option. As we can see in FIG. 2, 5 mg/kg MEAI gave better results in decreasing reward. The Y-axis in FIG. 2 represents time spent in the compartment that was less preferred during baseline testing. The behavioral data were analyzed with Ordinary one-way ANOVA. Data was expressed as mean±SEM and a probability value of *p<0.001 was considered significant. n=5-12 rats per group.

The K-Cluster method was used to partition the MEAI (5 mg/kg) values into two distinct groups. MEAI A represents values below 100 (seconds) and MEAI B represents values above 100 (seconds).

The many features and advantages of the present disclosure are apparent from the detailed specification, and thus it is intended by the appended claims to cover all such features and advantages of the present disclosure that fall within the true spirit and scope of the present disclosure. Further, since numerous modifications and variations will readily occur to those skilled in the art, it is not desired to limit the present disclosure to the exact construction and operation illustrated and described and accordingly, all suitable modifications and equivalents may be resorted to, falling within the scope of the present disclosure.

Moreover, those skilled in the art will appreciate that the conception upon which this disclosure is based may readily be used as a basis for designing other structures, methods, and systems for carrying out the several purposes of the present disclosure. Accordingly, the claims are not to be considered as limited by the foregoing description or examples.

We claim:

1. A method for treating cocaine addiction comprising administering to a subject in need thereof a therapeutically acceptable amount of a pharmaceutical composition comprising 5-methoxy-2-aminoindan or a pharmaceutically acceptable salt thereof, thereby treating the cocaine addiction.

2. The method according to claim 1, wherein the 5-methoxy-2-aminoindan or pharmaceutically acceptable salt thereof is administered as a daily dose of about 25 to about 100 mg.

3. The method according to claim 1, wherein the 5-methoxy-2-aminoindan or pharmaceutically acceptable salt thereof is administered as a daily dose of about 21 to about 84 mg.

4. The method according to claim 1, wherein the 5-methoxy-2-aminoindan or pharmaceutically acceptable salt thereof is administered as a daily dose of about 21 to about 100 mg, about 25 to about 90 mg, about 30 to about 80 mg, about 40 to about 70 mg, or about 50 to about 60 mg.

5. The method according to claim 2, wherein the daily dose is administered in a single dose or as more than one divided dose.

6. The method according to claim 1, wherein the 5-methoxy-2-aminoindan or a pharmaceutically acceptable salt thereof is administered twice a day.

7. The method according to claim 1, wherein the therapeutically effective amount comprises about 0.36 to about 1.4 mg/kg body weight/day, about 0.5 to about 1.3 mg/kg body weight/day, about 0.6 to about 1.2 mg/kg body weight/day, about 0.7 to about 1.1 mg/kg body weight/day, or about 0.8 to about 1.0 mg/kg body weight/day.

8. The method according to claim 1, wherein the cocaine addiction is a withdrawal symptom after withdrawal of cocaine.

9. The method according to claim 1, wherein the pharmaceutical composition further comprises at least one pharmaceutically acceptable carrier and/or excipient.

10. The method according to claim 9, wherein the pharmaceutical composition is a free-flowing powder, a tablet, a capsule, a lozenge, a liquid, a liquid concentrate, suspension, or a syrup.

11. The method according to claim 10, wherein the pharmaceutical composition is a unit dosage form composition.

12. The method according to claim 11, wherein an amount of 5-methoxy-2-aminoindan or pharmaceutically acceptable salt thereof in the unit dosage form is about 21 to about 100 mg, about 25 to about 90 mg, about 30 to about 80 mg, about 40 to about 70 mg, or about 50 to about 60 mg.

13. The method according to claim 12, wherein the amount of 5-methoxy-2-aminoindan or pharmaceutically acceptable salt thereof is about 50 mg.

14. The method according to claim 1, wherein administration of the pharmaceutical composition is oral, sublingual, buccal, vaginal, rectal, parenteral, transdermal, or by inhalation.

15. The method of claim 14, wherein the parenteral administration is intravenous, intramuscular, or subcutaneous.

16. The method according to claims 1-15, wherein treating the cocaine addiction attenuates craving for cocaine.

17. Use of a pharmaceutical composition comprising 5-methoxy-2-aminoindan or a pharmaceutically acceptable salt thereof for treating cocaine addiction according to claims 1-16.

18. A method for reducing the likelihood of a relapse of cocaine addiction comprising administering to a subject in need thereof a therapeutically acceptable amount of a pharmaceutical composition comprising 5-methoxy-2-aminoindan or a pharmaceutically acceptable salt thereof, thereby reducing the likelihood of a relapse of cocaine addiction.

19. The method according to claim 18, wherein the 5-methoxy-2-aminoindan or pharmaceutically acceptable salt thereof is administered as a daily dose of about 25 to about 100 mg.

20. The method according to claim 18, wherein the 5-methoxy-2-aminoindan or pharmaceutically acceptable salt thereof is administered as a daily dose of about 21 to 25 about 84 mg.

21. The method according to claim 18, wherein the 5-methoxy-2-aminoindan or pharmaceutically acceptable salt thereof is administered as a daily dose of about 21 to about 100 mg, about 25 to about 90 mg, about 30 to about 80 mg, about 40 to about 70 mg, or about 50 to about 60 mg.

22. The method according to claim 19, wherein the daily dose is administered in a single dose or as more than one divided dose.

23. The method according to claim 19, wherein the 5-methoxy-2-aminoindan or a pharmaceutically acceptable salt thereof is administered twice a day.

24. The method according to claim 18, wherein the therapeutically effective amount comprises about 0.36 to about 1.4 mg/kg body weight/day, about 0.5 to about 1.3 mg/kg body weight/day, about 0.6 to about 1.2 mg/kg body weight/day, about 0.7 to about 1.1 mg/kg body weight/day, or about 0.8 to about 1.0 mg/kg body weight/day.

25. The method according to claim 18, wherein the cocaine addiction is a withdrawal symptom after withdrawal of cocaine.

26. The method according to claim 18, wherein the pharmaceutical composition further comprises at least one pharmaceutically acceptable carrier and/or excipient.

27. The method according to claim 26, wherein the pharmaceutical composition is a free-flowing powder, a tablet, a capsule, a lozenge, a liquid, a liquid concentrate, suspension, or a syrup.

28. The method according to claim 27, wherein the pharmaceutical composition is a unit dosage form composition.

29. The method according to claim 28, wherein an amount of 5-methoxy-2-aminoindan or pharmaceutically acceptable salt thereof in the unit dosage form is about 21 to about 100 mg, about 25 to about 90 mg, about 30 to about 80 mg, about 40 to about 70 mg, or about 50 to about 60 mg.

30. The method according to claim 29, wherein the amount of 5-methoxy-2-aminoindan or pharmaceutically acceptable salt thereof is about 50 mg.

31. The method according to claim 18, wherein administration of the pharmaceutical composition is oral, sublingual, buccal, vaginal, rectal, parenteral, transdermal, or by inhalation.

32. The method of claim 31, wherein the parenteral administration is intravenous, intramuscular, or subcutaneous.

33. Use of a pharmaceutical composition comprising 5-methoxy-2-aminoindan or a pharmaceutically acceptable salt thereof for reducing the likelihood of a relapse of cocaine addiction according to claims 18-32.

34. A method for treating cocaine addiction comprising administering to a subject in need thereof a therapeutically acceptable amount of a pharmaceutical composition comprising 5-methoxy-2-aminoindan or a pharmaceutically acceptable salt thereof, and an N-acylethanolamine or a pharmaceutically acceptable salt thereof, thereby treating the cocaine addiction.

35. The method according to claim 34, wherein the 5-methoxy-2-aminoindan or pharmaceutically acceptable salt thereof is administered as a daily dose of about 25 to about 100 mg.

36. The method according to claim 34, wherein the 5-methoxy-2-aminoindan or pharmaceutically acceptable salt thereof is administered as a daily dose of about 21 to about 84 mg.

37. The method according to claim 34, wherein the 5-methoxy-2-aminoindan or pharmaceutically acceptable salt thereof is administered as a daily dose of about 21 to about 100 mg, about 25 to about 90 mg, about 30 to about 80 mg, about 40 to about 70 mg, or about 50 to about 60 mg.

38. The method according to claim 37, wherein the daily dose is administered in a single dose or in more than one divided dose.

39. The method according to claim 37, wherein the 5-methoxy-2-aminoindan or a pharmaceutically acceptable salt thereof is administered twice a day.

40. The method according to claim 34, wherein the therapeutically effective amount of the 5-methoxy-2-aminoindan or pharmaceutically acceptable salt thereof comprises about 0.36 to about 1.4 mg/kg body weight/day, about 0.5 to about 1.3 mg/kg body weight/day, about 0.6 to about 1.2 mg/kg body weight/day, about 0.7 to about 1.1 mg/kg body weight/day, or about 0.8 to about 1.0 mg/kg body weight/day.

41. The method according to claim 34, wherein the cocaine addiction is a withdrawal symptom after withdrawal of cocaine.

42. The method according to claim 34, wherein the pharmaceutical composition further comprises at least one pharmaceutically acceptable carrier and/or excipient.

43. The method according to claim 42, wherein the pharmaceutical composition is a free-flowing powder, a tablet, a capsule, a lozenge, a liquid, a liquid concentrate, suspension, or a syrup.

44. The method according to claim 43, wherein the pharmaceutical composition is a unit dosage form composition.

45. The method according to claim 44, wherein an amount of 5-methoxy-2-aminoindan or pharmaceutically acceptable salt thereof in the unit dosage form is about 21 to about 100 mg, about 25 to about 90 mg, about 30 to about 80 mg, about 40 to about 70 mg, or about 50 to about 60 mg.

46. The method according to claim 45, wherein the amount of 5-methoxy-2-aminoindan or pharmaceutically acceptable salt thereof is about 50 mg.

47. The method according to claim 34, wherein administration of the pharmaceutical composition is oral, sublingual, buccal, vaginal, rectal, parenteral, transdermal, or by inhalation.

48. The method of claim 47, wherein the parenteral administration is intravenous, intramuscular, or subcutaneous.

49. The method according to claims 34-48, wherein the N-acylethanolamine is palmitoylethanolamide or a pharmaceutically acceptable salt thereof.

50. The method according to claims 34-49, wherein the N-acylethanolamine or pharmaceutically acceptable salt thereof is administered as a daily dose of about 200 to about 1800 mg, about 250 to about 1550 mg, about 300 to about 1200 mg, about 350 to about 950 mg, about 400 to about 700 mg, about 450 to about 600 mg, or about 500 to about 550 mg.

51. The method according to claims 34-50, wherein treating the cocaine addiction attenuates craving for cocaine.

52. Use of a pharmaceutical composition comprising 5-methoxy-2-aminoindan or a pharmaceutically acceptable salt thereof, and palmitoylethanolamide or a pharmaceutically acceptable salt thereof, for treating cocaine addiction according to claims 34-51.

53. A method for reducing the likelihood of a relapse of cocaine addiction comprising administering to a subject in need thereof a therapeutically acceptable amount of a pharmaceutical composition comprising 5-methoxy-2-aminoindan or a pharmaceutically acceptable salt thereof, and an N-acylethanolamine or a pharmaceutically acceptable salt thereof, thereby reducing the likelihood of a relapse of cocaine addiction.

54. The method according to claim 53, wherein the 5-methoxy-2-aminoindan or pharmaceutically acceptable salt thereof is administered as a daily dose of about 25 to about 100 mg.

55. The method according to claim 53, wherein the 5-methoxy-2-aminoindan or pharmaceutically acceptable salt thereof is administered as a daily dose of about 21 to about 84 mg.

56. The method according to claim 53, wherein the 5-methoxy-2-aminoindan or pharmaceutically acceptable salt thereof is administered as a daily dose of about 21 to about 100 mg, about 25 to about 90 mg, about 30 to about 80 mg, about 40 to about 70 mg, or about 50 to about 60 mg.

57. The method according to claim 54, wherein the daily dose is administered in a single dose or in more than one divided dose.

58. The method according to claim 54, wherein the 5-methoxy-2-aminoindan or a pharmaceutically acceptable salt thereof is administered twice a day.

59. The method according to claim 53, wherein the therapeutically effective amount of the 5-methoxy-2-aminoindan or pharmaceutically acceptable salt thereof comprises about 0.36 to about 1.4 mg/kg body weight/day, about 0.5 to about 1.3 mg/kg body weight/day, about 0.6 to about 1.2 mg/kg body weight/day, about 0.7 to about 1.1 mg/kg body weight/day, or about 0.8 to about 1.0 mg/kg body weight/day.

60. The method according to claim 53, wherein the cocaine addiction is a withdrawal symptom after withdrawal of cocaine.

61. The method according to claim 53, wherein the pharmaceutical composition further comprises at least one pharmaceutically acceptable carrier and/or excipient.

62. The method according to claim 61, wherein the pharmaceutical composition is a free-flowing powder, a tablet, a capsule, a lozenge, a liquid, a liquid concentrate, suspension, or a syrup.

63. The method according to claim 62, wherein the pharmaceutical composition is a unit dosage form composition.

64. The method according to claim 63, wherein an amount of 5-methoxy-2-aminoindan or a pharmaceutically acceptable salt thereof in the unit dosage form is about 21 to about 100 mg, about 25 to about 90 mg, about 30 to about 80 mg, about 40 to about 70 mg, or about 50 to about 60 mg.

65. The method according to claim 64, wherein the amount of 5-methoxy-2-aminoindan or a pharmaceutically acceptable salt thereof is about 50 mg.

66. The method according to claim 53, wherein administration of the pharmaceutical composition is oral, sublingual, buccal, vaginal, rectal, parenteral, transdermal, or by inhalation.

67. The method of claim 66, wherein the parenteral administration is intravenous, intramuscular, or subcutaneous.

68. The method according to claims 53-67, wherein the N-acylethanolamine is palmitoylethanolamide or a pharmaceutically acceptable salt thereof.

69. The method according to claim 53-68, wherein the N-acylethanolamine or pharmaceutically acceptable salt thereof is administered as a daily dose of about wherein the N-acylethanolamine or pharmaceutically acceptable salt thereof is administered as a daily dose of about 200 to about 1800 mg, about 250 to about 1550 mg, about 300 to about 1200 mg, about 350 to about 950 mg, about 400 to about 700 mg, about 450 to about 600 mg, or about 500 to about 550 mg.

70. Use of a pharmaceutical composition comprising 5-methoxy-2-aminoindan or a pharmaceutically acceptable salt thereof, and an N-acylethanolamine or a pharmaceutically acceptable salt thereof, for reducing the likelihood of a relapse of cocaine addiction according to claims 53-69.
